(19) United States
(12) Patent Application Publication
(10) Pub. No.: US 2008/0160550 A1
(43) Pub. Date: Jul. 3, 2008

(54) Monitoring Enzyme-Substrate Reactions

(75) Inventor: Richard Gilbert, Chelmsford (GB)

Correspondence Address: SEED INTELLECTUAL PROPERTY LAW GROUP PLLC, 701 FIFTH AVE, SUITE 5400, SEATTLE, WA, 98104, US

(73) Assignee: E2V Biosensors Limited, Chelmsford (GB)

(21) Appl. No.: 11/665,650

(22) PCT Filed: Oct. 21, 2005
(86) PCT No.: PCT/GB05/04089

§ 371 Date: Feb. 6, 2008

(30) Foreign Application Priority Data
Oct. 22, 2004 (GB) 0423561.0

(51) Int. Cl. C12Q 1/37; C12Q 1/00; C12Q 1/48; C12M 1/00; C12Q 1/42

(52) U.S. Cl. 435/15; 435/4; 435/21; 435/23; 435/289.1

(57) ABSTRACT

Methods of electrochemically monitoring enzyme-substrate reactions are described. The substrate is tagged with a redox-active group. Modification of the substrate by an enzyme can then be monitored electrochemically by means of the redox-active group. Such methods can be used in particular to monitor non-redox enzyme reactions, such as kinase, phosphatase, or protease reactions, although redox enzyme en reactions can also be monitored by such methods. Screening assays to identify modulators of enzyme activity or new enzyme substrates are also described.

This invention relates to methods of monitoring enzyme-substrate reactions, to screening assays to identify modulators of enzyme activity or new enzyme substrates, and to substrates and electrochemical reaction chambers for use in such methods and assays.

Reactions catalysed by redox enzymes can be monitored electrochemically. See, for example, Barker P D, Hill H A. Prog Clin Biol Res. 1988;274:419-33: Direct electrochemical probes of redox protein and redox enzyme structure and function. Electrochemistry of cytochrome c is reviewed by Allen et al. (J. Electroanal. Chem. 178 (1984) 69-86), and Christensen and Hamnett (Techniques and Mechanisms in Electrochemistry (1994) 356-373). Electrochemical systems for assaying cytochrome P450 activity are described in WO 00/22158 and the references cited therein. Reactions catalysed by non-redox enzymes, however, cannot easily be monitored electrochemically because there is no obviously suitable chemical group that changes redox state as the reaction proceeds.

According to the invention it has been appreciated that non-redox enzyme reactions can be monitored electrochemically if the substrate has been tagged with a redox-active group. It has also been appreciated that use of tagged substrates is not limited to non-redox enzyme reactions. The invention may also be applied to redox enzymes, and so provides a new way of monitoring redox enzyme reactions.

According to the invention there is provided a method of monitoring modification of a substrate by an enzyme, the method comprising: providing a substrate that has been tagged with a redox-active group, and an enzyme that modifies the substrate; incubating the substrate with the enzyme under conditions for modification of the substrate by the enzyme; and electrochemically monitoring modification of the substrate by the enzyme by means of the redox-active group.

The redox-active group enables kinetic studies to be carried out on the enzyme using standard electrochemical techniques. For example, voltammetric wave profiles may be generated using methods such as linear sweep voltammetry (LSV) or cyclic voltammetry (CV) that are well known to those of ordinary skill in the art. The voltammetric wave profiles generated may be used, for example, to determine the catalytic activity of the enzyme, the rate of enzyme reaction, the kinetics of binding of a substrate or inhibitor to the enzyme, or to carry out competitive inhibition studies, measure thermodynamic responses, to screen for modulators of enzyme activity, or to measure drug cross-reactivity.

The term “redox-active group” is used herein to mean any group that can be tagged to the substrate and that is able to change its oxidation state (i.e. gain or lose electrons or protons) under conditions in which the enzyme is catalytically active. When a potential is applied and changed as a linear function of time, a change in oxidation state of the redox-active group tagged to the substrate will give rise to a voltammetric wave profile when current is plotted against applied electrode voltage.

There is also provided according to the invention a substrate that has been tagged with a redox-active group for use in a method of the invention.

The substrate may be tagged with the redox-active group by coupling the redox-active group to the substrate, preferably covalently. The redox-active group may be coupled directly to the substrate, or via a linker. Where a linker is used, the size and chemical nature of the linker should be chosen to minimise any interference of the linker with modification of the substrate by the enzyme.

The redox-active group should be chosen to give a good electrochemical signal under conditions in which the reaction to be monitored proceeds. The redox-active group should also be compatible with the chemical procedures required to couple it to the substrate.

Redox-active groups are well known to those of ordinary skill in the art. Examples of suitable groups include ferrocenes, fullerenes, and quinones.

The substrate may comprise or consist of a substrate corresponding to the full length natural substrate of the enzyme, or to only a part of the natural substrate, provided that the substrate used can be still be modified by the enzyme under appropriate conditions. For example, where the natural substrate of the enzyme is a protein or a peptide, the substrate used according to the invention may correspond to the full length protein or peptide, or to a fragment of the protein or peptide. The substrate may comprise or consist of a recombinant peptide or protein.

The substrate may be tagged with more than one redox-active group. The redox-active group(s) may be coupled to any suitable part of the substrate, as long as the redox-active group(s) does not interfere with modification of the substrate by the enzyme. Where the substrate is a peptide or protein, typically the redox-active group will be covalently coupled to an amino- and/or carboxy-terminal amino acid residue of the peptide or protein.

In preferred aspects of the invention the enzyme is a non-redox enzyme. According to particularly preferred aspects of the invention the enzyme is a kinase or a phosphatase. Where the enzyme is a kinase the substrate comprises an amino acid residue that is phosphorylated by the kinase under appropriate conditions. Where the enzyme is a phosphatase, the substrate comprises a phosphorylated amino acid residue that is de-phosphorylated by the phosphatase under appropriate conditions.

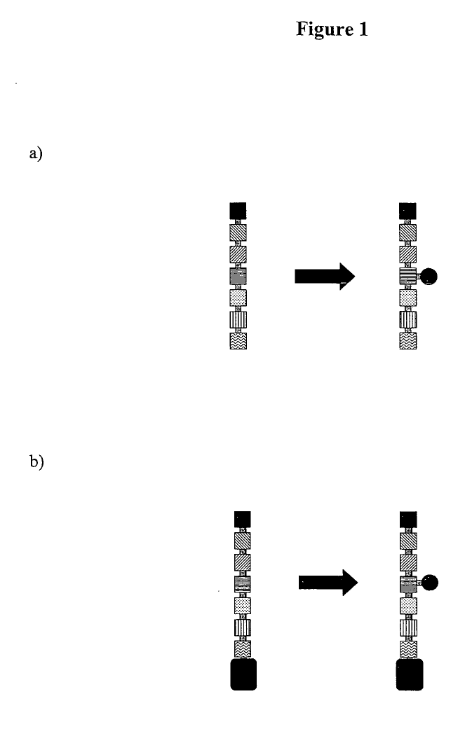

A kinase catalysed reaction is shown diagrammatically in FIG. 1(a). The squares represent amino acid residues of a substrate peptide chain. The kinase adds a phosphate (represented by a circle) to a particular amino acid residue, typically within a motif of 10-20 amino acid residues recognised specifically by the kinase. A phosphatase catalyses the reverse reaction.

Reactions catalysed by kinases and phosphatases cannot easily be followed electrochemically because there is no obviously suitable chemical group that can be used to follow the reaction. However, the reaction can be followed if the substrate molecule is tagged with a suitable redox-active group, such as a ferrocene, fullerene, or a quinone (FIG. 1 (b)). A phosphate group carries a double-negative charge, so addition or removal of this group changes the charge on the substrate by two. This causes a detectable change in the voltammetric wave profile of the substrate, and enables the amount of unconverted substrate to be quantified using standard voltammetric techniques (see Example 1 below).

The substrate that is tagged with the redox-active group may be an entire substrate protein, or correspond to a fragment of a substrate protein recognised by the kinase or phosphatase. For example, the substrate may comprise the amino acid sequence of a motif recognised specifically by the enzyme.

The kinase or phosphatase substrate may be tagged with more than one redox-active group. In one embodiment, the substrate is tagged with a first redox-active tag at the amino-terminal end, and a second redox-active group at the carboxy-terminal end of the substrate.

Where the enzyme is a kinase, preferably the redox-active group(s) tagged to the substrate forms an electronic coupling with a phosphate group that is added by the kinase. Where the enzyme is a phosphatase, preferably the redox-active group(s) tagged to the substrate forms an electronic coupling with a phosphate group of the substrate that is removed by the phosphatase. Formation of an electronic coupling between the phosphate group and the redox-active group is expected to increase the difference between voltammetric wave profiles obtained for the unconverted substrate and the fully converted product. This should increase the accuracy with which the amount of unconverted substrate is quantified.

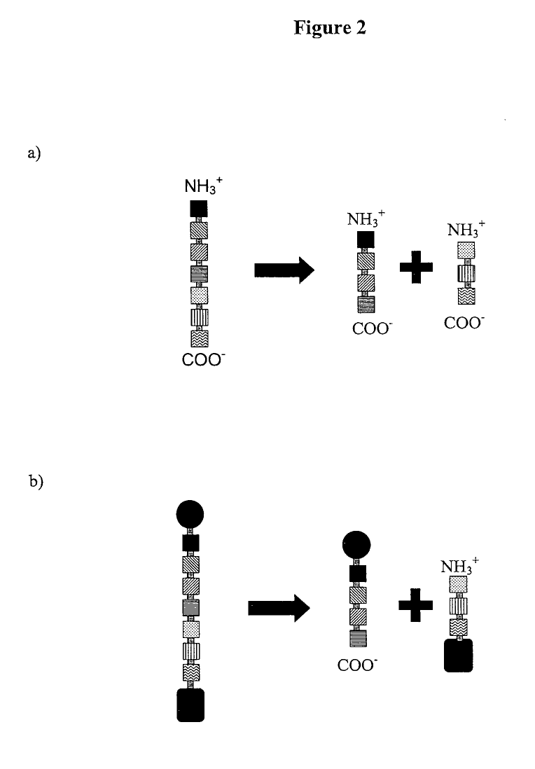

In another particularly preferred aspect of the invention the enzyme is a protease that cleaves the substrate at a cleavage site under appropriate conditions. Proteases catalyse essentially the same reaction, shown diagrammatically in FIG. 2(a). Under physiological conditions, the substrate and reaction products adopt a zwitterionic state with a positive charge at the amino-terminus and a negative charge at the carboxy-terminus, but retain an overall charge of zero (neglecting any side-chain charges, which remain constant between the left and right-hand sides of the reaction).

Reactions catalysed by proteases cannot readily be followed electrochemically since there are no obviously suitable chemical groups within the substrate and products that change their redox state as the reaction proceeds. However, the reaction can be monitored electrochemically if the substrate peptide is tagged at its amino- and carboxy-terminus with a redox-active group (FIG. 2(b)). The uncleaved tagged substrate still has an overall charge of zero, but the cleavage products do not. One has an additional positive charge and the other has an additional negative charge.

The voltammetric wave profiles of the cleavage products are substantially different to the voltammetric wave profile of the substrate. This enables the amount of cleaved substrate to be quantified using standard voltammetry techniques (see Example 2 below).

In other embodiments of this aspect of the invention the protease substrate may instead be tagged with one or more redox-active groups away from the amino- and carboxy-terminus (so that the positive charge at the amino terminus, and the negative charge at the carboxy terminus remain). However, it is preferred that the redox-active group(s) are at the amino- and/or carboxy-terminus of the substrate because this is expected to improve the discrimination between the uncleaved substrate and the cleavage products.

In other embodiments of this aspect of the invention, the protease substrate may be tagged with a single redox-active group. However, it is preferred that the protease substrate is tagged with a redox-active group either side of the cleavage site so that both cleavage products are then detectable electrochemically.

Any electronic or through-space energetic coupling that can be engineered between the redox-active groups is expected to further enhance the differences between the voltammetric wave profiles obtained for the substrate and cleavage products and, therefore, the accuracy with which the amount of cleaved products can be quantified.

The first and second redox-active groups may be the same chemical groups, but are preferably different chemical groups to maximise the change in detectable electrochemical signal when the substrate is cleaved.

According to other aspects of the invention, the enzyme is a redox enzyme. Such aspects provide a new way of monitoring redox enzyme reactions. An advantage of such aspects is that the enzyme reaction is monitored directly (by monitoring the voltammetric wave profile of the substrate) rather than indirectly as in conventional methods (by monitoring the voltammetric wave profile of a reagent, such as NAD⁺/NADH, other than the enzyme or substrate).

In some embodiments, it may be desirable to provide a redox-active group-tagged substrate that can be recognised by more than one enzyme. For example, a peptide substrate may be provided that has amino acid sequence comprising different motifs recognised by different enzymes of the same class, such as different kinase enzymes. This enables electrochemical studies to be carried out on the different enzymes using the same substrate.

In other embodiments, a redox-active group-tagged substrate peptide is provided that can be recognised by enzymes of two or more different classes, such as kinase, phosphatase, and protease enzymes. This enables electrochemical studies to be carried out on the different classes of enzyme using the same substrate.

Methods of the invention may be used for kinetic studies of the enzyme, or to identify a modulator of the activity of the enzyme, or to identify a substrate for the enzyme.

According to the invention there is provided a screening assay for identifying a modulator of the activity of an enzyme, the assay comprising:

providing a substrate that has been tagged with a redox-active group, and an enzyme that modifies the substrate;

incubating the tagged substrate with the enzyme under conditions for modification of the substrate by the enzyme;

electrochemically monitoring modification of the substrate by the enzyme by means of the redox-active tag in the presence of a candidate modulator of the activity of the enzyme; and

determining whether the candidate modulator modulates the activity of the enzyme.

Typically, it is determined whether the candidate modulator modulates the activity of the enzyme by comparing modification of the substrate by the enzyme in the presence of the candidate modulator with modification of the substrate by the enzyme in the absence of the candidate modulator.

The candidate modulator may be an inhibitor, an activator, or an enhancer of the activity of the enzyme.

There is also provided according to the invention a screening assay for identifying a substrate of an enzyme, the assay comprising:

providing an enzyme and a candidate substrate for the enzyme that has been tagged with a redox-active group;

incubating the enzyme with the candidate substrate under conditions that allow modification by the enzyme of a known substrate for the enzyme; and

determining electrochemically by means of the redox-active tag whether the enzyme modifies the candidate substrate.

There is further provided according to the invention a method of making a substrate that has been tagged with a redox-active group, which comprises coupling a redox-active group to a substrate.

Coupling procedures compatible with automated peptide synthesisers are preferred, since tagged substrates can easily be produced using such procedures. Fmoc (9-fluorenylmethylcarbonyl) or t-Boc (t-Butoxycarbonyl) coupling reactions are particularly preferred.

There is also provided according to the invention an electrochemical reaction chamber comprising a substrate for an enzyme, the substrate having been tagged with a redox-active group, and optionally an enzyme capable of modifying the substrate.

According to the invention there is further provided a kit for monitoring modification of a substrate by an enzyme, the kit comprising a substrate that has been tagged with a redox-active group, and an enzyme that modifies the substrate.

Important advantages of methods of the invention are that they can be automated, and performed using microfluidic electrochemical sensor devices. This allows high throughput processes to be carried out, such as high throughput screening of candidate modulators of enzyme activity, or simultaneous collection of data for the same enzyme-substrate reaction under several different conditions (for example serial dilutions of an inhibitor of the enzyme).

Methods of the invention may be particularly useful for secondary screening of candidate drug compounds previously identified by high throughput screening of compound libraries to have some activity against an enzyme. Secondary screening can be used to confirm the activity, measure the potency, and assess the selectivity of the candidate drug compounds. Most secondary screens used during drug discovery are performed manually and so consume significant resources. Methods of the invention allow high throughput, automated secondary screening to be carried out.

Methods of the invention may also be used for lead optimisation of candidate drug compounds to identify those compounds with the best safety and efficacy profiles. Again, high throughput, automated lead optimisation can be carried out using methods of the invention.

It is even possible that methods of the invention could be used for high throughput, automated primary screening of libraries of compounds to identify candidate drug compounds that are active against an enzyme.

Embodiments of the invention are now described in the following examples, with reference to the accompanying drawings in which:

FIG. 1(a) shows a diagrammatic representation of a kinase reaction;

FIG. 1(b) shows a diagrammatic representation of a kinase reaction in which the substrate has been tagged with a redox-active group;

FIG. 2(a) shows a diagrammatic representation of a protease reaction;

FIG. 2(b) shows a diagrammatic representation of a protease reaction in which the amino- and carboxy-terminal ends of the substrate have been tagged with a redox-active group;

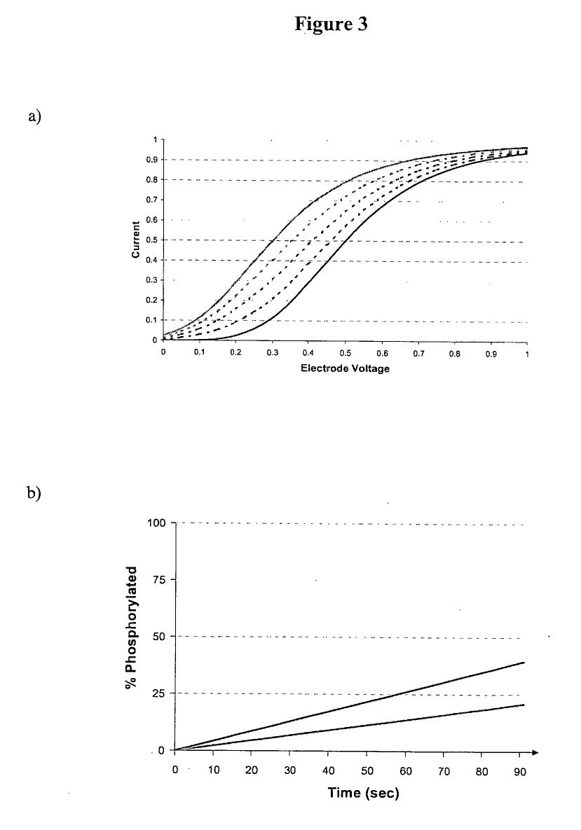

FIG. 3(a) shows the expected change in voltammetric wave profile for a kinase reaction according to an embodiment of the invention;

FIG. 3(b) shows the expected effect of a kinase inhibitor on kinase reaction rate;

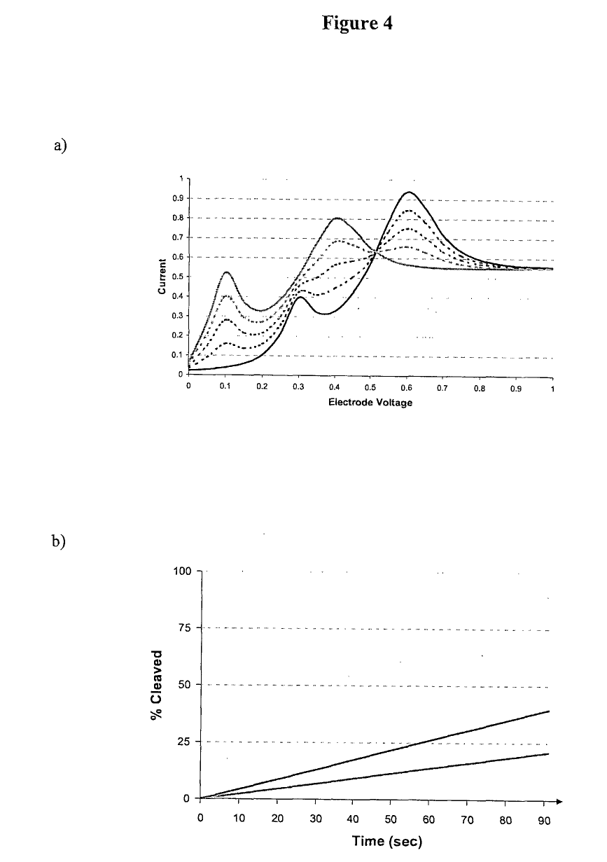

FIG. 4(a) shows the expected change in voltammetric wave profile for a protease reaction according to an embodiment of the invention; and

FIG. 4(b) shows the expected effect of a protease inhibitor on protease reaction rate.

### EXAMPLE 1

### Electrochemical Kinase/Phosphatase Assay

A peptide substrate for the tyrosine kinase, c-Src, tagged with a redox-active ferrocene at (i) its amino-terminus, or (ii) its carboxy-terminus is shown below:

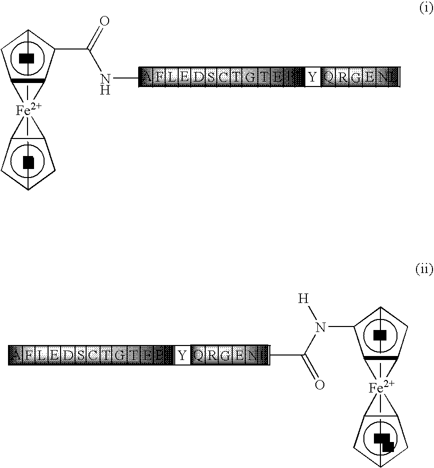

The tyrosine residue that is phosphorylated by c-Src is represented by the white box. Provided that all of the side chains of the tagged substrate are ionised under the assay conditions, phosphorylation of the tyrosine residue will change the total charge on the tagged substrate from −1 to −3.

To electrochemically monitor modification of the tagged substrate by c-Src, the reaction is performed in aqueous solution (at a pH, temperature, and ionic concentration that closely resembles physiological conditions) in the presence of ATP in an electrochemical reaction chamber. A potential is applied to the electrochemical reaction chamber (using a working electrode and a reference electrode), and a current response is measured (using the working electrode and an auxiliary electrode). Linear sweep voltammetry is used to generate a voltammogram. FIG. 3(a) shows a hypothetical voltammogram that could be obtained.

Even though there may not be a direct transfer of charge between the phosphate and the redox tag, the overall change in molecular charge will change the attractive or repulsive force it experiences when approaching an electrode. Therefore, although the shape of the wave plot might not change, it will shift ‘sideways’ with an amount proportional to the degree of phosphorylation of the substrate peptide (see FIG. 3(a)).

As the peptide changes from fully non-phosphorylated to fully phosphorylated, the wave curve in the voltammogram shifts between two well-defined limits (illustrated by the light and dark solid lines in FIG. 3(a)). This response is used to follow the degree to which the peptide is phosphorylated, and so can be used to monitor the rate of the reaction catalysed by the enzyme.

The enzyme might normally have a reaction rate shown by the upper, dark line in FIG. 3(b). If a candidate drug is able to inhibit this reaction, then the amount of modified substrate produced in a given time will be reduced, shown by the lower, pale line in FIG. 3(b). Provided there is always a large excess of substrate peptide available, this method of following the reaction is particularly robust, since the ratio of the values of the two lines at any given time should remain constant, and the accuracy of the result can be improved simply by running the assay for longer.

### EXAMPLE 2

### Electrochemical Protease Assay

A peptide substrate for hepatitis NS3 protease, tagged with ferrocene and fullerene is shown below (the site at which the protease cleaves the substrate is marked with an arrow):

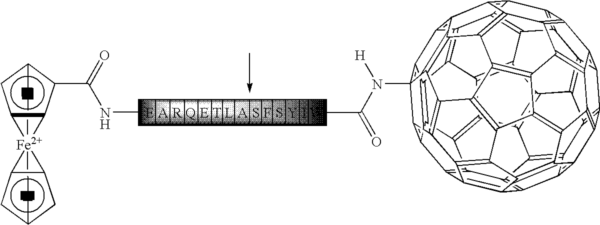

Cleavage of the substrate by the protease generates two products, one with a positive charge at its amino terminus, and the other with a negative charge at its carboxy terminus.

To electrochemically monitor cleavage of the tagged substrate by the NS3 protease, the reaction is performed in aqueous solution (at a pH, temperature, and ionic concentration that closely resembles physiological conditions) in an electrochemical reaction chamber. A potential is applied to the electrochemical reaction chamber (using a working electrode and a reference electrode), and a current response is measured (using the working electrode and an auxiliary electrode). Linear sweep voltammetry is used to generate a voltammogram. FIG. 4(a) shows a hypothetical voltammogram that could be obtained.

Even though there may not be a direct transfer of charge between the two redox tags, the cleavage products will experience substantially different attractive or repulsive force compared to the substrate when approaching an electrode. Therefore, the shape of the wave plot will change by an amount proportional to the degree to which the substrate is cleaved (see FIG. 4(a)).

As the substrate is cleaved, the curve in the voltammogram will shift between limits defined by the electrochemical profiles of the substrate and cleavage products (see the light and dark lines in FIG. 4(a)). This response can be used to follow the degree to which the substrate is cleaved, and so can be used to monitor the rate of the reaction catalysed by the enzyme.

The enzyme might normally have a reaction rate shown by the upper, dark line in FIG. 4(b). If a candidate drug is able to inhibit this reaction, then the amount of substrate cleaved in a given time will be reduced, shown by the lower, pale line in FIG. 4(b). Again, provided there is always a large excess of substrate available, this method of following the reaction is particularly robust, since the ratio of the values of the two lines at any given time should remain constant, and the accuracy of the result can be improved simply by running the assay for longer. Although the actual slopes of the lines might vary, the ratio is determined by the candidate drug's behaviour and is independent of enzyme concentration. This means that the sensor with which the assay is carried out has a high tolerance to manufacturing variation.

Full binding kinetics studies can be performed using the approaches described in Examples 1 and 2 simply by changing the concentration of the candidate drug. This sort of experiment could, for example, involve making a dilution series along a row of microtitre plate wells, and using a 24-probe chip design to collect 24 data points simultaneously, thereby generating a full kinetics dataset in a single measurement. Other kinetics studies such as competitive inhibition, thermodynamic responses and drug cross-reactivity can just as readily be performed by mixing drug candidates or changing the reaction temperature, pH, etc.

1. A method of monitoring modification of a substrate by an enzyme, the method comprising:
(a) incubating (i) a substrate that has been tagged with at least one redox-active group, with (ii) an enzyme that modifies the substrate, under conditions for modification of the substrate by the enzyme; and
(b) electrochemically monitoring modification of the substrate by the enzyme by means of the redox-active group.

2. The method according to claim 1, wherein the enzyme is a non-redox enzyme.

3. The method according to claim 2, wherein the non-redox enzyme is a kinase.

4. The method according to claim 2, wherein the non-redox enzyme is a phosphatase.

5. The method according to claim 2, wherein either (i) the non-redox enzyme is a kinase and the redox-active group forms an electronic coupling with a phosphate group of the substrate that is added by the kinase, or (ii) the non-redox enzyme is a phosphatase and the redox-active group forms an electronic coupling with a phosphate group of the substrate that is removed by the phosphatase.

6. The method according to claim 2, wherein the non-redox enzyme is a protease.

7. The method according to claim 6, wherein the substrate comprises a peptide that is tagged with a first redox-active group at an amino-terminal end of the peptide, and a second redox-active group at a carboxy-terminal end of the peptide.

8. The method according to claim 7, wherein the first and second redox-active groups are different chemical groups.

9. The method according to claim 1, wherein the redox-active group is a ferrocene, a fullerene, or a quinone.

10. The method according to claim 1, wherein modification of the substrate by the enzyme is monitored in the presence and absence of a modulator of substrate-modifying activity of the enzyme.

11. The method according to claim 10, wherein modification of the substrate by the enzyme is monitored in the presence of two or more different concentrations of the modulator.

12. (canceled)

13. A screening assay method for identifying a modulator of substrate-modifying activity of an enzyme, comprising:
(a) incubating, in the presence and, absence of a candidate modulator, (i) a substrate that has been tagged with a redox-active group with (ii) an enzyme that modifies the substrate, under conditions for modification of the substrate by the enzyme;
(b) electrochemically monitoring modification of the substrate by the enzyme in the absence and presence of the candidate modulator, by means of the redox-active group; and
(c) determining from the electrochemically monitored modification of (b) whether the candidate modulator modulates the activity of the enzyme, and therefrom identifying the modulator of substrate-modifying activity of the enzyme.

14. The screening assay method according to claim 13, wherein modification of the substrate by the enzyme in the presence of the candidate modulator is compared with modification of the substrate by the enzyme in the absence of the candidate modulator.

15. The assay according to claim 13, wherein the candidate modulator is a candidate inhibitor of substrate-modifying activity of the enzyme.

16. A screening assay method for identifying a substrate of an enzyme, comprising:
(a) incubating (i) an enzyme and (ii) a candidate substrate for the enzyme wherein the candidate substrate has been tagged with a redox-active group, under conditions that allow modification by the enzyme of a known substrate for the enzyme; and
(b) determining electrochemically by means of the redox-active group that the enzyme modifies the candidate substrate, and therefrom identifying the candidate substrate as a substrate of the enzyme.

17. A substrate that has been tagged with at least one redox-active group and that is selected from the group consisting of:
(i) a substrate the modification of which can be electrochemically monitored by means of the redox-active group,
(ii) a substrate the modification of which by an enzyme that modifies the substrate can be electrochemically monitored by means of the redox-active group,
(iii) a substrate the modification of which by a non-redox enzyme that modifies the substrate can be electrochemically monitored by means of the redox-active group, wherein the non-redox enzyme is a kinase or a phosphatase,
(v) a substrate the modification of which by a kinase can be electrochemically monitored by means of the redox-active group, wherein the redox-active group forms an electronic coupling with a phosphate group of the substrate that is added by the kinase,
(vi) a substrate the modification of which by a phosphatase can be electrochemically monitored by means of the redox-active group, wherein the redox-active group forms an electronic coupling width a phosphate group of the substrate that is removed by the phosphatase,
(vii) a substrate the modification of which by a protease that modifies the substrate can be electrochemically monitored by means of the redox-active group,
(viii) a substrate the modification of which by a protease that modifies the substrate can be electrochemically monitored, by means of the redox-active group, wherein the substrate comprises a peptide that is tagged with a first redox-active group at an amino-terminal end, of the peptide, and a second redox-active group at a carboxy-terminal end of the peptide,
(ix) a substrate the modification of which by a protease that modifies the substrate can be electrochemically monitored by means of the redox-active group, wherein substrate comprises a peptide that is tagged with a first redox-active group at an amino-terminal end, of the peptide, and a second redox-active groups at a carboxy-terminal end of the peptide, wherein the first and second redox-active groups are different chemical groups, and
(x) the substrate of any one of (i)-(ix) wherein the redox-active, group is a ferrocene, a fullerene, or a quinone.

18. The method of any one of claims 1, 13 and 16 wherein the substrate comprises the substrate according to claim 17.

19. A method of making a substrate that has been tagged with a redox-active group, which comprises coupling a redox-active group to a substrate.

20. An electrochemical reaction chamber, comprising:
(a) a substrate for an enzyme, the substrate having been tagged with a redox-active group; and at least one of:
(b) an enzyme capable of modifying the substrate; and
(c) one or more of a working electrode, a reference electrode and an auxiliary electrode.

21. A kit for monitoring modification of a substrate by an enzyme, the kit comprising a substrate that has been tagged with a redox-active group, and an enzyme that modifies the substrate.
